(19) United States
(12) Patent Application Publication
(10) Pub. No.: US 2021/0330874 A1
(43) Pub. Date: Oct. 28, 2021

(54) Multicolumn Blood Plasma Immunomodulating Agents Rebalancing System

(71) Applicant: Thompson Oncology Devices LLC, Arvada, CO (US)

(72) Inventors: Joel Wallace Graff, Butte, MT (US); Christopher Armstrong Kay, Fort Collins, CO (US); Randall Jay Daughenbaugh, Rapid City, SD (US)

(73) Assignee: Thompson Oncology Devices LLC, Arvada, CO (US)

(21) Appl. No.: 17/236,888

(22) Filed: Apr. 21, 2021

Related U.S. Application Data
(60) Provisional application No. 63/014,419, filed on Apr. 23, 2020.

(51) Int. Cl. A61M 1/36; A61M 1/34

(52) U.S. Cl. CPC A61M 1/362; A61M 1/3496; A61M 1/3403; A61M 2202/0415; A61M 2205/0238; A61M 2202/0445; A61M 2230/30; A61M 2230/06; A61M 2230/42; A61M 2230/20; A61M 2230/50

(57) ABSTRACT

Generally, a blood plasma immunomodulating agent rebalancing system. In particular, a blood plasma sTNFR and cytokine rebalancing system and methods of rebalancing sTNFR and cytokines in the blood plasma of a subject during plasmapheresis.

This United States Non-Provisional Patent Application claims the benefit of U.S. Provisional Patent Application No. 63/014,419, filed Apr. 23, 2020, hereby incorporated by reference herein.

### I. FIELD OF THE INVENTION

Generally, a blood plasma immunomodulating agent rebalancing system. In particular, a blood plasma sTNFR and cytokine rebalancing system and methods of rebalancing sTNFR and cytokines in the blood plasma of a subject during plasmapheresis.

### II. SUMMARY OF THE INVENTION

A broad object of embodiments of the invention can be to provide a blood plasma soluble tumor necrosis factor receptor (“sTNFR”) and cytokine rebalancing system (or the “system”). The sTNFR and cytokine rebalancing system can include a plasmapheresis device coupled to a valved conduit configured to receive blood plasma from said plasmapheresis device. The valved conduit can include a valve assembly operable to direct the blood plasma of the subject to a sTNFR removal medium or operable to direct the blood plasma of the subject to a cytokine removal medium. The valved conduit operable to direct blood plasma passed through the sTNFR removal medium to the subject or operable to direct the blood plasma passed through the cytokine removal medium to the subject, wherein the sTNFR removal medium removes sTNFRs, and in particular embodiments, selectively removes soluble tumor necrosis factor receptor type I (“sTNFR1”) or soluble tumor necrosis factor receptor type II (“sTNFR2”), or the combination thereof, in the blood plasma of the subject, and the cytokine removal medium removes cytokines in the blood plasma of the subject, whereby the system rebalances levels of sTNFR, or selectively sTNFR1 or sTNFR2, or combinations thereof, and levels of cytokines in the blood plasma of the subject.

Another broad object of embodiments of the invention can be to provide a method of treating or alleviating symptoms of a disorder in a subject, including one or more of decreasing levels of sTNFR, or selectively sTNFR1 or sTNFR2, or combinations thereof, in blood plasma of a subject by contact of the blood plasma with a sTNFR, sTNFR1 or sTNR2 removal medium, wherein decreasing levels of sTNFR, sTNFR1 or sTNFR2 in the blood plasma of the subject can result in increasing levels of cytokines in said blood plasma of the subject, thereafter, reducing contact of the blood plasma of the subject having increased levels of cytokines with the sTNFR, sTNFR1 or sTNFR2 removal medium and contacting the blood plasma of the subject having increased levels of cytokines with a cytokine removal medium, thereby decreasing blood plasma levels of the cytokines in the blood plasma of the subject, and altering blood plasma level of said cytokines in said blood plasma of said subject toward or to normative values.

Naturally, further objects of the invention are disclosed throughout other areas of the specification, drawings, photographs, and claims.

### III. A BRIEF DESCRIPTION OF THE DRAWINGS

FIG. 1 is a block flow diagram of a blood plasma sTNFR and cytokine rebalancing system and methods of rebalancing sTNFR and cytokines in blood plasma of a subject during plasmapheresis.

FIG. 2 is a graph illustrating results of using a particular embodiment of the system having a valved conduit by which blood plasma of a subject undergoing plasmapheresis can be operated to direct blood plasma to a sTNFR removal medium resulting in decreasing levels of sTNFR1 or sTNFR2 in the blood plasma and thereafter exhibiting increasing levels of cytokines TNF-α, IL-6, and IL-1β in the blood plasma over time, during which the valve conduit can be operated to direct blood plasma to a cytokine removal medium resulting in subsequent decreasing levels of cytokines TN-α, IL-6, and IL-1β in the blood plasma over time. Levels of sTNFR1 or sTNFR2, and levels of cytokines TNF-α, IL-6, and IL-1β in the blood plasma over can be measured by enzyme-linked immunosorbent assay (ELISA).

### IV. DETAILED DESCRIPTION OF THE PREFERRED EMBODIMENTS

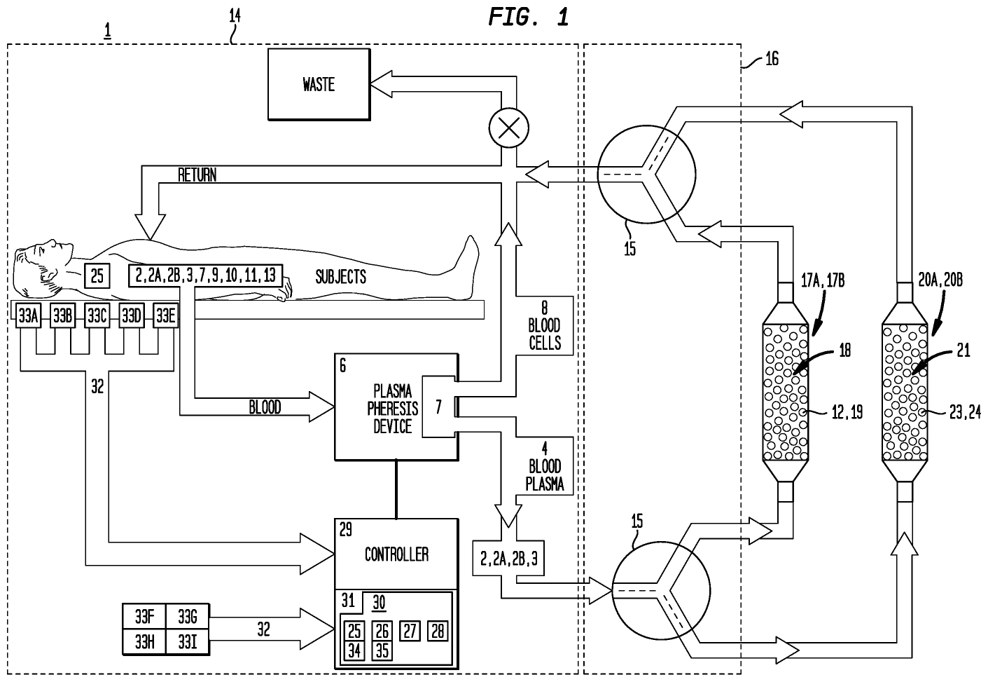

Now with primary reference to FIG. 1, which provides illustrative examples of embodiments of a blood plasma sTNFR and cytokine rebalancing system (1) and methods of rebalancing sTNFR (2) and cytokines (3) in blood plasma (4) of a subject (5) during plasmapheresis (4).

Embodiments may be described in terms of an apparatus or a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a function, a procedure, a subroutine, a subprogram, or the like. Furthermore, the apparatus and processes disclosed herein may be implemented in one or a combination of system components, computer hardware or software, or combinations thereof. If the process is implemented, in whole or in part, in software, functions may be stored or transmitted as one or more instructions or code contained on a non-transitory computer-readable medium. Computer-readable media includes both computer storage media and communication media including any medium that facilitates transfer of a computer program from one place to another.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed or that the first element must precede the second element in some manner. In addition, unless stated otherwise, a set of elements may comprise one or more elements.

As such, the particular embodiments or elements of the invention disclosed by the description or shown in the figures or tables accompanying this application are not intended to be limiting, but rather illustrative of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

The term “plasmapheresis device (6)” for the purposes of this invention broadly includes any device adapted to process the blood (7) of a subject (5) to separate the blood plasma (4) from the blood cells (8). The blood plasma (4) can be replaced with another solution such as saline or albumin, or the blood plasma (4) can be treated and then returned to the subject (5) in accordance with embodiments of the invention, and without sacrificing the breadth of the foregoing, illustrative examples of plasmapheresis devices (6) useful in embodiments of the invention can be Cobe Spectra, Fenwal Aurora or the Fresenius COM.TEC.

The term “blood plasma (4)” for the purposes of this invention means the liquid part of the blood (7) and lymphatic fluid, which makes up about half of the volume of blood. Blood plasma (4) while substantially devoid or devoid of cells contains antibodies and other proteins, and without sacrificing the breadth of the foregoing, blood plasma contains tumor necrosis factor α (“TNF-α”) (9), tumor necrosis factor receptor 1 (“TNFR1”) (10) and tumor necrosis factor receptor 2 (“TNFR2”) (11) and cytokines (3).

The term “tumor necrosis factor α” or “TNF-α” (9) for the purposes of this invention means a multifunctional cytokine mediating pleiotropic biological functions in both health and disease states. TNF-α (9) is secreted primarily by monocytes and macrophages but can also be secreted by other cell types. The list of processes regulated by TNF-α (9) is extensive, and includes inflammation, immunoregulation, cytotoxicity and antiviral effects. Vilcek et al, J. Biol. Chem., 266:7313-7316 (1991), and without sacrificing the breadth of the foregoing, TNF-α (9) plays an integral role in destroying tumors, mediating responses to tissue injury, and protecting hosts from infections by various microorganisms. Vassali, Ann. Rev. Immunol., 10:411-452 (1992).

Recent evidence also implicates TNF-α (9) activity in the pathogenesis of many infections. TNF is thought to play a central role in the pathophysiological consequences of Gram-negative sepsis and endotoxic shock, including fever, malaise, anorexia, and cachexia. Beutler et al., Nature 316:552-554 (1985). TNF-α (9) has also been implicated in the pathogenesis of a variety of diseases and disorders. These pathologies may result from the aberrant regulation of TNF activity, in which the pathologies manifest as a result of excessive or insufficient TNF activity. Among the activities for which TNF-α (9) is most noted are its pro-inflammatory actions, sometimes termed the “acute phase immune response.” Unfortunately, if not properly regulated, these proinflammatory responses can result in tissue injury and chronic inflammatory diseases, such as rheumatoid arthritis, inflammatory bowel disease, septic shock, cachexia, autoimmune disorders, graft-versus-host disease and insulin resistance. Piguet et al., J. Exp. Med., 166:1280 (1987).

Insufficient TNF activity can result in the detrimental effects of an exaggerated immune response demonstrated in some of these diseases, exemplified by overstimulation of interleukin-6 and granulocyte/macrophage-colony stimulating factor (GM-CSF) secretion, enhanced cytotoxicity of polymorphonuclear neutrophils, prolonged expression of cellular adhesion molecules, induction of procoagulant activity on vascular endothelial cells, increased adherence of neutrophils and lymphocytes, and stimulation of the release of platelet activating factor from macrophages, neutrophils and vascular endothelial cells. Vassali, Ann. Rev. Immunol., 10:411-452 (1992).

The term “tumor necrosis factor receptor 1” (“TNFR1”) (10) and “tumor necrosis factor receptor 2” (“TNFR2”) (11) for the purposes of this invention means two transmembrane receptors, the 55 kilodalton Type I receptor (also written as “CD120a,” and referred to herein as “TNFR1”) (10) (GenBank accession number X55313 for human TNFR1) and the 75 kilodalton Type II receptor (11) (also written as “CD120b,” and referred to herein as “TNFR2”) (GenBank accession number NM_001066 for human TNFR2), and without sacrificing the breadth of the foregoing, both TNFR1 (10) and TNFR2 (11) demonstrate strong affinity for TNF-α (9), although, these two receptors demonstrate no apparent homology in their cytoplasmic domains. This fact is consistent with the observation that these two receptors transduce different signals to the nucleus via distinct signaling intermediates. Lewis et al., Proc. Natl. Acad. Sci. USA 88:2830-2834 (1991). TNFR1 (10) and TNFR2 (11) comprise the extracellular domains of TNF receptors derived by proteolytic cleavage of the transmembrane forms. Engelmann et al., J. Biol. Chem., 264:11974-11980 (1989).

In the case of the TNFR1 (10), this proteolytic activity results in the cleavage and shedding of the extracellular N-terminal domain as soluble tumor necrosis factor receptor 1 (“sTNFR1”) (2A) having an affinity for TNF-α (9) that is similar to that of intact membrane receptors. Due to this affinity, the free receptors are able to bind and sequester TNF-α (9), thereby inhibiting the biological action of TNF-α (9), which can suppress TNF-α (9) signaling by reducing the number of functional TNF-α(9) receptors acting at the cell membrane and by competitive binding of TNF-α (9). Pfeffer et al., Cell 73:457-467 (1993).

In the case of the TNFR2 (10), this proteolytic activity results in the cleavage and shedding of a protein with residues 23-257, which terminates immediately before the transmembrane region, and a protein with residues 23-185. U.S. Pat. No. 5,945,397. Both sTNFR1 (2A) and sTNFR2 (2B) fragments are soluble and capable of binding TNF-α (9).

The term “TNF-α mutein (12)” refers to a wild type TNF-α mutein (12) or a variant having one or more amino acid substitutions relative to a parent sequence and retaining specific binding activity for a sTNFR1 (2A) or sTNFR2 (2B), or combinations thereof. Examples of TNF-α muteins (12) include the human TNF-α muteins (12) and in particular embodiments, designated muteins 1, 2, 3, 4, 5 and 6, or combinations thereof described in U.S. Pat. No. 8,501,918. Analogous muteins of species other than human are similarly included, for example, muteins analogous to muteins 1, 2, 3, 4, 5 or 6 in the other mammalian species, or combinations thereof.

The term “cytokines (3)” for the purpose of this invention means a category of small proteins (about 5 kDa to about 50 kDa) involved in autocrine, paracrine and endocrine signaling as immunomodulating agents. The Cytokine Handbook (ed. Angus W. Thomson), 3^{rd} edition, Academic Press, San Diego, p. 35-72 (1998). Without sacrificing the breadth of the foregoing, cytokines (3) include chemokines, interferons, interleukins, lymphokines, and tumor necrosis factors.

The term “cytokine release syndrome (“CRS) (13)” for the purposes of this invention means a systemic inflammatory response by aberrant regulation of TNF-α (9) activity resulting in the massive release of a range of cytokines (3) that overwhelms counter-regulatory homeostatic mechanisms and results in a cytokine storm that can have detrimental effects on the patient, and without sacrificing the breadth of the foregoing, IL-6, IL-10, and interferon (“IFN”) are among the core cytokines (3) that are consistently found to be elevated in serum of subjects with CRS (13). Mild symptoms of CRS (13) include fever, fatigue, headache, rash, arthralgia, and myalgia. More severe cases are characterized by hypotension as well as high fever and can progress to an uncontrolled systemic inflammatory response with vasopressor-requiring circulatory shock, vascular leakage, disseminated intravascular coagulation, and multi-organ system failure.

Removal of soluble TNF-α receptors sTNFR 1 (2A) or sTNFR2 (2B) from the blood plasma (4) of a subject (5) during TNF-α (9) based therapies allowing TNF-α (9) to act on membrane bound TNFR1 (10) and TNFR2 (11) allows TNF-α (9) signaling to occur in tumor vasculature, tumor stromal cells, and cancer cells. TNF-α (9) signaling in these cells can result in tumor regression as result of removing sTNFR1 (2A) and sTNFR2 (2B) from the blood plasma (4). Increased TNF-α (9) signaling resulting from removal of soluble TNF-α receptors sTNFR 1 (2A) and sTNFR2 (2B) from the blood plasma (4) and increased levels of TNF-α (9) in the blood (7) of a subject (7) may result in CRS (13).

The term “subject (5)” for the purposes of this invention means a human or non-human animal, and without sacrificing the breadth of the foregoing, can include a human or non-human animal displaying pathology resulting from abnormal cytokine activity, a subject suspected of displaying pathology resulting from abnormal cytokine activity, and a subject at risk of displaying pathology resulting from abnormal cytokine activity.

The term “rebalancing” for the purposes of this invention means altering the respective levels of sTNFRs and cytokines in the blood plasma of a subject undergoing plasmapheresis by redirecting the blood plasma between a sTNFR removal medium and a cytokine removal medium which may be used to treat, alleviate symptoms of, reduce the risk of or prevent a pathology resulting from abnormal cytokine activity including but not necessarily limited to CRS.

Now, with primary reference to FIG. 1, embodiments of a blood plasma sTNFR and cytokine rebalancing system (1) and methods of rebalancing sTNFR (2) and cytokines (3) in blood plasma (4) of a subject (5) during plasmapheresis (14) can include a valved conduit (15) adapted to receive blood plasma (4) from a plasmapheresis device (6). The valved conduit (15) can include a valve assembly (16) operable to reconfigure the flow path of the valved conduit (15) to direct the blood plasma (4) to a first column (17A) or a first stirred reactor (17B) or other structure, or a combination thereof, containing or allowing the blood plasma (4) to contact a sTNFR removal medium (18). In particular embodiments, the sTNFR removal medium can specifically bind sTNFR1 (2A) or sTNFR2 (2B), or combinations thereof.

The sTNFR removal medium (18) can, but need not necessarily, comprise a plurality of TNF-α muteins (12) immobilized on a first extracorporeal biocompatible solid support (19). As illustrative examples, the solid support (19) can comprise one or more of: macroporous beads include, but are not limited to, naturally occurring materials such as agarose, cellulose, controlled pore glass, or synthetic materials such as polyacrylamide, cross-linked agarose (as illustrative examples Trisacryl™, Sephacryl™, and Ultrogel™), azlactone, polymethacrylate, polystyrene/divinylbenzene; non-porous beads including, but are not limited to, silica, polystyrene, latex; hollow fibers and membranes composed of natural or synthetic materials where natural materials include, but are not limited to, cellulose and modified cellulose, for example, cellulose diacetate or triacetate, and synthetic materials include, but are not limited to, polysulfone, polyvinyl, polyacetate, and combinations thereof.

The plurality of TNF-α muteins (12) immobilized on the extracorporeal biocompatible solid support (18), whether directly or via an additional molecule attached to the solid support, can have one or more binding sites capable of selectively binding to one or more sTNFR1 (2A) or sTNFR2 (2B) with an affinity sufficient to remove all or a portion of the sTNFR1 (2A) or sTNRF2 (2B) from the blood plasma (4) or other biological fluid passed through the first column (17A) or stirred reactor (17B). In particular embodiments. the plurality of TNF-α muteins (12) can be covalently attached to the extracorporeal biocompatible solid support (18), or alternately through high-affinity, non-covalent interaction with an additional molecule which has been covalently attached to the solid support (18). For example, a biotinylated binding partner can interact with avidin or streptavidin previously conjugated to the inert medium. The blood plasma (4) having all or a portion of the sTNFR1 (2A) or TNRF2 (2B) removed can be returned to the subject (5).

Again, with primary reference to FIG. 1, embodiments of a blood plasma sTNFR and cytokine rebalancing system (1), can further allow operation the valve assembly (17) to reconfigure the flow path of the valved conduit (15) to direct the blood plasma (4) to a second column (20A) or second stirred reactor (20B) containing or allowing the blood plasma (4) to contact a cytokine removal medium (21). The cytokine removal medium (21) can comprise a second extracorporeal biocompatible solid support (22) having an affinity sufficient to remove all or a portion of the cytokines (3) from the blood plasma (4) or other biological fluid passed through the second column (20). As an illustrative example the cytokine removal medium (21) can comprise a porous polymer sorbent (23) can comprise or consist of one or more of: a polystyrene divinylbenzene polymer, a polystyrene divinylbenzene and polyvinyl pyrrolidone copolymer, or a polystyrene divinylbenzene polymer coated with polyvinylpyrrolidone, and combinations thereof. The porous polymer sorbent (23) can comprise porous particles (24) which can be configured as one or more of spherical, ellipsoidal, rod, prismatic, or the like.

The porous particles (24) can have a diameter occurring in a range of about 200 microns to about 1000 microns. In particular embodiments, the diameter can be selected from the group consisting of: about 250 micron to about 350 micron, about 300 micron to about 400 micron, about 350 micron to about 450 micron, about 400 micron to about 500 micron, about 450 micron to about 550 micron, about 500 micron to about 600 micron, about 550 micron to about 650 micron, about 600 micron to about 700 micron, about 650 micron to about 750 micron, about 700 micron to about 800 micron, about 750 micron to about 850 micron, about 800 micron to about 900 micron, about 850 micron to about 950 micron, and combinations thereof.

The porous particles (24) can have density of about 1.0 g cm⁻³ and porosity occurring in a range of about 50.0% to about 75.0% with a pore size (25) occurring in a range of about 5 Angstrom units to about 80 Angstrom units.

In particular embodiments, the polymer sorbent (23) can adsorb cytokines of less than 50 kDa while excluding absorption of larger proteins, for example, albumin (70 kDa) or fibrinogen (340 kDa). The polymer sorbent (23) can adsorb, as examples one or more of: interleukin 6 (“IL-6”), interleukin 10 (“IL-10”), high mobility group box protein 1 (“HMGB-1”), interleukin 8 (“IL-8”), interleukin 18 (“IL-18”), monocyte chemoattractant protein 1 (“MCP-1”), interleukin 2 (“IL-2”), interleukin (“IL-1β”) and calcium binding peptide S100B (“S100B”).

As illustrative examples, cytokine removal medium (21) suitable for use in embodiments of the invention can comprise or consist of: polystyrene divinylbenzene copolymer and polyvinyl pyrrolidone polymers available from MedaSorb Technologies, Inc. (Princeton, N.J.) the trademarks CYTOSORB and BETASORB.

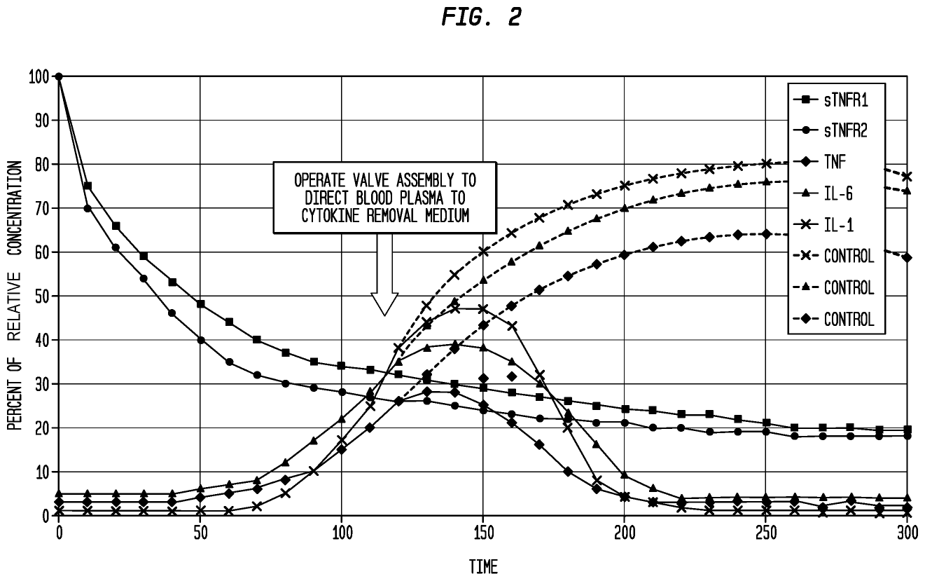

Now, with primary reference to FIGS. 1 and 2, in particular embodiments, the condition of the subject (5) can be monitored during the time course of plasmapheresis (14) by monitoring or sensing measurable variables (25) of the subject (5) undergoing plasmapheresis (14) which indicate the level of cytokine activity (27), including, but not necessarily, CRS (13). Measurable variables (25) of the subject (5) indicative of levels of cytokine activity (27) or CRS (13) can comprise or consist of: temperature, blood pressure, heart rate, respiratory rate, oxygen saturation, white blood cell count, cardiac output, lactate level, cytokines interleukin-6, interleukin-10, and TNF-α, or combinations thereof.

In the absence of indicators of elevated levels of cytokine activity (27), the valve assembly (16) can be operated to deliver blood plasma (4) of the subject (5) through the valved conduit (15) from the plasmapheresis device (6) to the to the sTNFR removal medium (18) to remove sTNFR, and in certain embodiments, specifically bind sTNFR1 (2A) or sTNFR2 (2B), and return the blood plasma (4) depleted of or reduced in sTNFR, sTNFR1 (2A) or sTNFR2 (2B), or combinations thereof, to the subject (5). Upon indications of elevated cytokine activity (27), the valve assembly (16) can be operated to deliver blood plasma (4) of the subject (5) through the valved conduit (15) to the cytokine removal medium (21) to remove cytokines (3) and return blood plasma (4) depleted or reduced in cytokines (3) until the measurable variables (25) of the subject (5) indicate an alleviation of or acceptable reduction in cytokine activity (27), including but not necessarily limited to alleviation of symptoms of CRS (13). In particular embodiments, the measurable variables (25) may be associated with a pre-selected threshold values (26) indicative of elevated cytokine activity (27) and upon exceeding the pre-selected threshold value (26) of one or more measurable variables (25) triggers reconfiguration of the valved conduit (15) from a first valved conduit configuration which delivers blood plasma (4) of the subject (5) through the valved conduit (15) from the plasmapheresis device (6) to the to the sTNFR removal medium (18) to a second valved conduit configuration which delivers blood plasma (4) of the subject (5) through the valved conduit (15) to the cytokine removal medium (21). Measurable variables (25) may later be associated with cytokine activity (27) below the pre-selected threshold value (26) of such one or more measurable variables (25) to trigger reconfiguration of the valved conduit (15) from the second valved conduit configuration to the first valved conduit configuration which delivers blood plasma (4) of the subject (5) through the valved conduit (15) from the plasmapheresis device (6) to the to the sTNFR removal medium (18). Reconfiguration of the valved conduit (15) between the first and second valved configurations can be repeated as necessary during the time course of plasmapheresis (14).

Again, with primary reference to FIG. 1, in particular embodiments, the valve assembly (16) can be operated to reconfigure the valved conduit (15) to deliver blood plasma (4) from the subject (5) to the sTNFR removal medium (18) for a first pre-selected period of time (28A) and then operated to reconfigure the valved conduit (15) to deliver blood plasma (4) from the subject (5) to the cytokine removal medium (21) for a second preselected period of time (28B). The first and second pre-selected period of time (28A, 28B) can have the same or different time duration and the reconfiguration of the valved conduit (15) repeated throughout the time course of the plasmapheresis (14) which occur in range of about 4 hours to 96 hours.

In particular embodiments, the sTNFR removal medium (18) or the cytokine removal medium (20) can, but need not necessarily, be exchanged for a fresh sTNFR removal medium (18) or the cytokine removal medium (20) at periodic intervals between about 4 hours and about 96 hours during treatment of a subject (5) by plasmapheresis (14), depending upon the application.

Now, with primary reference to FIG. 2 and Table 1, reconfiguration of the valved conduit (15) to deliver the blood plasma (4) to the sTNFR removal medium (18) can over the time course of continuous plasmapheresis (14) substantially reduce the level of sTNFR. In an illustrative example of FIG. 2, the relative levels of sTNFR1 or sTNFR2, or combinations thereof, can be reduced by about 80 percent over a time course of 300 minutes of plasmapheresis (14). Notably, depletion or reduction of sTNFR1 or TNFR2 in the blood plasma (4) can cause a substantial increasing cytokine activity (27). In the illustrative example of FIG. 2, the relative cytokine activity (27) of TMFα, IL-6 and IL-1β begins to substantially increase at about 50 minutes during the time course of plasmapheresis (14). The increase in cytokine activity (27) whether monitored or sensed can trigger reconfiguration of the valved conduit (15) to deliver blood plasma (4) to the cytokine removal medium (20) to deplete or reduce levels of the cytokines (3) in the blood plasma over the time course of plasmapheresis (14). In the illustrative example of FIG. 2, the reconfiguration of the valved conduit (15) to deliver blood plasma (4) to the cytokine removal medium (20) occurs in the range of 100 minutes to about 125 minutes during the course of plasmapheresis (14). Subsequently, the level of cytokines (3) continues to increase in the blood plasma (4) until about 150 minutes in the time course of plasmapheresis (14) and then become rapidly depleted or reduced in the interval from about 150 minutes to about 225 minutes in the time course of plasmapheresis (14). In the illustrative example of FIG. 2, the level of cytokines (3) in the blood plasma can return to substantially the same level preceding plasmapheresis (14).

Referring to Table 1, the table sets out the numerical values of percentages corresponding to the plots set out in FIG. 2.

TABLE 1

| Time | sTNFR1 | STNFR2 | TNF-α | IL-6 | IL-1β |
|---|---|---|---|---|---|
| 0 | 100 | 100 | 3 | 5 | 1 |
| 10 | 75 | 70 | 3 | 5 | 1 |
| 20 | 66 | 61 | 3 | 5 | 1 |
| 30 | 59 | 54 | 3 | 5 | 1 |
| 40 | 53 | 46 | 3 | 5 | 1 |
| 50 | 48 | 40 | 4 | 6 | 1 |
| 60 | 44 | 35 | 5 | 7 | 1 |
| 70 | 40 | 32 | 6 | 8 | 2 |
| 80 | 37 | 30 | 8 | 12 | 5 |
| 90 | 35 | 29 | 10 | 17 | 10 |
| 100 | 34 | 28 | 15 | 22 | 17 |
| 110 | 33 | 27 | 20 | 28 | 25 |
| 120 | 32 | 26 | 26 | 35 | 38 |
| 130 | 31 | 26 | 28 | 38 | 44 |
| 140 | 30 | 25 | 28 | 39 | 47 |
| 150 | 29 | 24 | 25 | 38 | 47 |
| 160 | 28 | 23 | 21 | 35 | 43 |
| 170 | 27 | 22 | 16 | 30 | 32 |
| 180 | 26 | 22 | 10 | 23 | 20 |
| 190 | 25 | 21 | 6 | 16 | 8 |
| 200 | 24 | 21 | 4 | 9 | 4 |
| 210 | 24 | 20 | 3 | 6 | 3 |
| 220 | 23 | 20 | 3 | 4 | 2 |
| 230 | 23 | 19 | 3 | 4 | 1 |
| 240 | 22 | 19 | 3 | 4 | 1 |
| 250 | 21 | 19 | 3 | 4 | 1 |
| 260 | 20 | 18 | 3 | 4 | 1 |
| 270 | 20 | 18 | 2 | 4 | 1 |
| 280 | 20 | 18 | 3 | 4 | 1 |
| 290 | 19 | 18 | 2 | 4 | 1 |
| 300 | 19 | 18 | 2 | 4 | 1 |

In studies in which blood plasma (4) during plasmapheresis (14) was measured before and subsequent to delivery of the blood plasma (4) to the cytokine removal media (20), cytokine (3) level in subjects (5) can show significant reduction of interleukin-6 plasma levels (7977.27 pg/mL to 210.18 pg/mL, p=0.0077) and interleukin-10 plasma levels (from 687.19 pg/mL to 36.95 pg/mL, p=0.0180). In those patients with detectable TNF-α (9) plasma level, its reduction may not be significant (p=0.138). The median removal ratio can be about 80% for interleukin-6, about 90% for interleukin-10, and about 29% for TNF-α.

Now, with primary reference to FIG. 1, embodiments, can but need not necessarily, include a controller (29) having a program code (30) contained in a non-transitory computer readable medium (31). Execution of the program code (30) can control operation of one or more of: the plasmapheresis device (6) or the valve assembly (16) which reconfigures the flow path of the valved conduit (15) to direct blood plasma (4) from the subject (5) to either or both of the sTNFR removal medium (18) and the cytokine removal medium (21) whether based on pre-selected time periods (28) or monitored measurable variables (25) and return the blood plasma (4) to the subject (5). However, inclusion of a controller (29) is not intended to preclude embodiments in which measured variables (25) and operation of the valve assembly (16) occurs manually.

Again, with primary reference to FIG. 1, in particular embodiments, the controller (29) can further operate to receive data (32) from one or more sensing devices (33) sensing measurable variables (25) of the subject (5) undergoing plasmapheresis (14). The sensing devices (33) can be associated with the subject (5) undergoing plasmapheresis (14) and can include one or more of a temperature sensor (33A) to measure body temperature, a heart rate monitor (33B) to measure heart rate, a pulse oximeter (33C) to measure oxygen saturation, a respiration sensor (33D) to measure respiration rate, blood pressure sensor (33E) to measure blood pressure. Additionally, additional measurable variables (25) of the subject (5) can be monitored during the course of plasmapheresis (14) including white blood cell count (33F), cardiac output (33G), lactate level (33H), cytokines levels (33I) by ELISA including IL-6, IL-10, and TNF-α, or combinations thereof.

Again, with primary reference to FIGS. 1 and 2 and Table 1, embodiments of the controller (29) can process the data (32) associated with measurable variables (25) monitored by one or more sensing devices (33, 33A, 33B, 33C, 33D, 33E) or additional measurable variables (25) of the subject (5) including one or more of white blood cell count (33F), cardiac output (33G), lactate level (33H), or cytokine levels (33I) obtained by ELISA including IL-6, IL-10, and TNF-α, or combinations thereof, to generate measured variable values (34) which can be compared to pre-selected threshold values (26) associated with each measurable variable (25) as part of the program code (30) or a threshold value database (35). Based on one or a combination of the determined measured variable values (34) occurring below the pre-selected threshold values (26), the controller (29) can operate the valve assembly (16) to direct blood plasma (4) from the subject (5) to the sTNFR removal medium (18) to deplete or reduce sTNFR1 (2A) or sTNFR2 (2B), or combinations thereof, in the blood plasma (4) of the subject (5) undergoing plasmapheresis (14)(as shown in the example of FIG. 2 and Table 1). Upon exceeding one or a combination of preselected threshold values (26) for one or a combination of the measurable variables (25), the controller (29) can operate the valve assembly (16) to direct blood plasma (4) from the subject (5) to the cytokine removal medium (21) to deplete or reduce cytokines (3) in the blood plasma (4) of the subject (5) undergoing plasmapheresis (14) (as shown in the example of FIG. 2 and Table 1).

Again, with primary reference to FIGS. 1 and 2, in particular embodiments, the valve assembly (16) can be configured to deliver the blood plasma (4) generated by the plasmapheresis device (6) concurrently to the sTNFR removal medium (18) and the cytokine removal medium (21) the blood plasma (4) from both the sTNFR removal medium (18) and the cytokine removal medium (21) and thereafter admixed and returned to the subject (5) undergoing plasmapheresis (14).

Each embodiment of the inventive a blood plasma sTNFR and cytokine rebalancing system (1) and methods of rebalancing sTNFR and cytokines in the blood plasma of a subject during plasmapheresis provides a substantial advantage in treating or alleviating symptoms of a disorder during TNF-α (9) based therapies allowing TNF-α (9) to act on membrane bound TNFR1 (10) and TNFR2 (11) to allow TNF-α (9) signaling to occur in tumor vasculature, tumor stromal cells, and cancer cells while periodically or concurrently antagonizing the resultant increasing level of cytokines (3) to prevent or reduce systemic inflammatory response, including, but not limited to CRS (13) which can overwhelm counter-regulatory homeostatic responses with the substantial advantage of preventing or reducing tissue injury in the subject (5) undergoing plasmapheresis (14) TNF-α (9) based therapies.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “controller” should be understood to encompass disclosure of the act of “controlling”—whether explicitly discussed or not—and, conversely, were there effectively disclosure of the act of “controlling”, such a disclosure should be understood to encompass disclosure of a “controller” and even a “means for controlling.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in Merriam-Webster's Collegiate Dictionary, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the multicolumn blood plasma immunomodulating agents rebalancing systems herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. An apparatus, comprising:
a plasmapheresis device;
a valved conduit adapted to receive blood plasma from said plasmapheresis device, said valved conduit including:
a valve assembly operable to direct said blood plasma to a soluble TNF receptors removal medium or operable to direct said blood plasma to a cytokine removal medium, said valve assembly operable to direct said blood plasma passed through said TNF receptors removal medium to said subject or operable to direct said blood plasma passed through said cytokine removal medium to said subject,
wherein said soluble TNF receptor removal medium adapted to remove soluble TNF receptors in blood plasma of a subject,
wherein said cytokine removal medium adapted to remove cytokines in said blood plasma of said subject, whereby said apparatus rebalances levels of said soluble TNF receptors and said cytokines toward normative levels in said blood plasma of said subject.

2. The apparatus of claim 1, wherein said soluble TNF receptors removal medium comprises a plurality of TNF-α muteins immobilized on an extracorporeal biocompatible solid support.

3. The apparatus of claim 2, where said plurality of TNF-α muteins are covalently attached to said extracorporeal biocompatible solid support.

4. The apparatus of claim 3, wherein said extracorporeal biocompatible solid support contained in a first column fluidically coupled to said valved conduit.

5. The apparatus of claim 3, wherein said extracorporeal biocompatible solid support contained in a first reactor fluidically coupled to said valved conduit.

6. The apparatus of claim 2, wherein each of said plurality of TNF-α muteins immobilized on the extracorporeal biocompatible solid support have a single binding site capable of selectively binding to one of said soluble TNF receptors with an affinity sufficient to deplete the soluble TNF receptors from the biological fluid.

7. The apparatus of claim 2, wherein said plurality of TNF-α muteins comprises one or more of: TNF-α mutein 1, TNF-α mutein 2, TNF-α mutein 3, TNF-α mutein 4, TNF-α mutein 5, TNF-α mutein 6, and combinations thereof.

8. The apparatus of claim 2, wherein said plurality of TNF-α muteins is selected from the group of TNF-α muteins consisting of: TNF-α mutein 1, TNF-α mutein 2, TNF-α mutein 3, TNF-α mutein 4, TNF-α mutein 5, TNF-α mutein 6, and combinations thereof.

9. The apparatus of claim 1, wherein said soluble TNF receptors comprise soluble TNF receptors type 1 or soluble TNF receptors type 2.

10. The apparatus of claim 9, wherein said soluble TNF receptors comprises soluble TNF receptor type 1 and soluble TNF receptor type 2.

11. The apparatus of claim 1, wherein said cytokine removal medium non-specifically binds said cytokines.

12. The apparatus of claim 11, wherein said cytokine removal medium adsorbs cytokines of less than 50 kDa.

13. The apparatus of claim 12, wherein said cytokines of less than 50 kDa include one or more of IL-6, IL-10, HMGB-1, IL-8, IL-18, MCP-1, IL-2, IL-1β and S100B.

14. The apparatus of claim 11, wherein said cytokine removal medium which non-specifically binds said cytokines comprises a polystyrene-divinylbenzene copolymer beads with a biocompatible polyvinylpyrrolidone coating.

15. The apparatus of claim 14, wherein said a polystyrene-divinylbenzene copolymer beads with said biocompatible polyvinylpyrrolidone coating comprises CYTOSORB copolymer beads.

16. The apparatus of claim 11, wherein said cytokine removal medium which non-specifically binds said cytokines comprises a 2-methacryloyloxyethyl phosphorylcholine bead.

17. The apparatus of claim 16, wherein said 2-methacryloyloxyethyl phosphorylcholine bead comprises MEDASORB beads.

18. The apparatus of claim 1, wherein said a valve assembly manually operated by a user of said apparatus to direct said blood plasma to said soluble TNF receptor removal medium or to direct said blood plasma to said cytokine removal medium.

19. The apparatus of claim 18, wherein said user operates said valve assembly between said soluble TNF receptor removal medium and said cytokine removal medium based on monitoring one or more measurable variables of said subject indicative of levels of cytokine activity.

20. The apparatus of claim 19, where said measurable variables of said subject include one or more of temperature, blood pressure, heart rate, respiratory rate, oxygen saturation, white blood cell count, cardiac output, lactate level, interleukin-6, interleukin-10, and TNF-α.

21. The apparatus of claim 1, further comprising:
one or more sensors which correspondingly generate a sensor signal which varies based on change in a measurable variable indicative of levels of cytokine activity in said blood plasma of said subject;
a controller which operates to:
convert said sensor signal to a measured value of said measurable variable;
compare said measured value to pre-selected threshold value of said measured variable of cytokine activity in said blood plasma of said subject;
identify said measured value in excess of said pre-selected threshold value of said measured variable of cytokine activity in said blood plasma of said subject;
actuate said valve assembly to direct said blood plasma to said cytokine removal medium to decrease levels of said cytokines in said blood plasma of said subject.

22. The apparatus of claim 19, wherein said controller actuates said valve assembly to direct said blood plasma to said soluble TNF receptor removal medium to decrease levels of said soluble TNF receptor in said blood plasma which increases levels of and said cytokines in said blood plasma of said subject.

23-32. (canceled)
